SEQUENCE LISTING (42 sequences)

SEQ ID NO: 1 - protein, 651 aa, Phanerochaete chrysosporium [sequence omitted]

SEQ ID NO: 2 - protein, 576 aa, Schwanniomyces vanrijiae
MVQSHYDFVIVGGGTAGNTVAGRLAENPNVTVLVVEAGVANSADLPEITTPSNAMNLRGSKHDWAYKTTLVKRDDYERIEKPNTRGKALGGSSSLNYFTWIPGCKPTFDRWAEYGGEEWTWDPLVPYLRKSATYHDDTGLYNPELKKLGAGGPIPISHSELVEELEPFRENLIKAWKSTGKPFTENIYDGEMIGLNHCISTIYHGKRSGSFLFVKNRPNITIIPEVHSKNLIIDASNTAKGVVVIDKEGNEHSFYATREVILSQGVFESPKLLMLSGVGPRKELESNGIEVKVESRHVGQNLLDHPGVPFVLQVKDDICVDDILMRQNEKNKAAHVQYQKDGSGPVGSGLLELVGFPRIDEYFEKDPLYRERKAANGGKDPFCPEGQPHFELDFVGMYGTAFQWHFPTPKKGSHITIVVDLVRPVSEGGEVTLNSADPLEQPKINLNFFADELDIVGMREGIRFTYDLLTKGDGFKDLVVKEFPWEMPLDDDKEMRRAVLDRCQTAFHPCGTNRLSKNIEQGVVDPALKVHGVKNLRVIDASIIPVIPDCRIQNSVYMIGEKGADLIKAAHKDLYN

SEQ ID NO: 3 - protein, 400 aa, Pseudomonas putida
MAGNKSVVYHGTRDLRVETVPYPKLEHNNRKLEHAVILKVVSTNICGSDQHIYRGRFIVPKGHVLGHEITGEVVEKGSDVELMDIGDLVSVPFNVACGRCRNCKEARSDVCENNLVNPDADLGAFGFDLKGWSGGQAEYVLVPYADYMLLKFGDKEQAMEKIKDLTLISDILPTGFHGCVSAGVKPGSHVYIAGAGPVGRCAAAGARLLGAACVIVGDQNPERLKLLSDAGFETIDLRNSAPLRDQIDQILGKPEVDCGVDAVGFEAHGLGDEANTETPNGALNSLFDVVRAGGAIGIPGIYVGSDPDPVNKDAGSGRLHLDFGKMWTKSIRIMTGMAPVTNYNRHLTEAILWDQMPYLSKVMNIEVITLDQAPDGYAKFDKGSPAKFVIDPHGMLKNKL

SEQ ID NO: 4 - protein, 771 aa, Artificial Sequence
  Synthetic
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGHMGHPEEVDVIVCGGGPAGCVVAGRLAYADPTLKVMLIEGGANNRDDPWVYRPGIYVRNMQRNGINDKATFYTDTMASSYLRGRRSIVPCANILGGGSSINFQMYTRASASDWDDFKTEGWTCKDLLPLMKRLENYQKPCNNDTHGYDGPIAISNGGQIMPVAQDFLRAAHAIGVPYSDDIQDLTTAHGAEIWAKYINRHTGRRSDAATAYVHSVMDVQDNLFLRCNARVSRVLFDDNNKAVGVAYVPSRNRTHGGKLHETIVKARKMVVLSSGTLGTPQILERSGVGNGELLRQLGIKIVSDLPGVGEQYQDHYTTLSIYRVSNESITTDDFLRGVKDVQRELFTEWEVSPEKARLSSNAIDAGFKIRPTEEELKEMGPEFNELWNRYFKDKPDKPVMFGSIVAGAYADHTLLPPGKYITMFQYLEYPASRGKIHIKSQNPYVEPFFDSGFMNNKADFAPIRWSYKKTREVARRMDAFRGELTSHHPRFHPASPAACKDIDIETAKQIYPDGLTVGIHMGSWHQPSEPYKHDKVIEDIPYTEEDDKAIDDWVADHVETTWHSLGTCAMKPREQGGVVDKRLNVYGTQNLKCVDLSICPDNLGTNTYSSALLVGEKGADLIAEELGLKIKTPHAPVPHAPVPTGRPATQQVR

SEQ ID NO: 5 - protein, 33 aa, Phanerochaete chrysosporium
  misc_feature (4)..(4): Xaa can be any naturally occurring amino acid
  misc_feature (21)..(21): Xaa can be any naturally occurring amino acid
  misc_feature (26)..(26): Xaa can be any naturally occurring amino acid
  misc_feature (31)..(31): Xaa can be any naturally occurring amino acid
GRRXIVPCANILGGGSSINFXMYTRXSASDXDD

SEQ ID NO: 6 - protein, 6 aa, Phanerochaete chrysosporium
  misc_feature (5)..(5): Xaa can be any naturally occurring amino acid
LLPLXK

SEQ ID NO: 7 - protein, 6 aa, Phanerochaete chrysosporium
QDFLRA

SEQ ID NO: 8 - protein, 6 aa, Phanerochaete chrysosporium
TAHGAE

SEQ ID NO: 9 - protein, 5 aa, Phanerochaete chrysosporium
GRRSD

SEQ ID NO: 10 - protein, 11 aa, Phanerochaete chrysosporium
  misc_feature (6)..(8): Xaa can be any naturally occurring amino acid
LPGVGXXXQDH

SEQ ID NO: 11 - protein, 11 aa, Phanerochaete chrysosporium
  misc_feature (3)..(3): Xaa can be any naturally occurring amino acid
  misc_feature (9)..(9): Xaa can be any naturally occurring amino acid
AGXKIRPTXEE

SEQ ID NO: 12 - protein, 5 aa, Phanerochaete chrysosporium
KPDKP

SEQ ID NO: 13 - protein, 8 aa, Phanerochaete chrysosporium
  misc_feature (5)..(5): Xaa can be any naturally occurring amino acid
LEYPXSRG

SEQ ID NO: 14 - protein, 11 aa, Phanerochaete chrysosporium
  misc_feature (4)..(4): Xaa can be any naturally occurring amino acid
  misc_feature (7)..(7): Xaa can be any naturally occurring amino acid
YKKXREXARRM

SEQ ID NO: 15 - protein, 8 aa, Phanerochaete chrysosporium
  misc_feature (3)..(3): Xaa can be any naturally occurring amino acid
GEXTSHHP

SEQ ID NO: 16 - protein, 7 aa, Phanerochaete chrysosporium
  misc_feature (5)..(5): Xaa can be any naturally occurring amino acid
EEDDXAI

SEQ ID NO: 17 - protein, 10 aa, Phanerochaete chrysosporium
  misc_feature (6)..(6): Xaa can be any naturally occurring amino acid
ETTWHXLGTC

SEQ ID NO: 18 - protein, 14 aa, Phanerochaete chrysosporium
  misc_feature (4)..(4): Xaa can be any naturally occurring amino acid
  misc_feature (9)..(9): Xaa can be any naturally occurring amino acid
  misc_feature (11)..(11): Xaa can be any naturally occurring amino acid
DLSXCPDNXGXNTY

SEQ ID NO: 19 - protein, 584 aa, Artificial Sequence
  Synthetic
MVQSHYDFVIVGGGTAGNTVAGRLAENPNVTVLVVEAGVANSADLPEITTPSNAMNLRGSKHDWAYKTTLVKRDDYERIEKPNTRGKALGGSSSLNYFTWIPGCKPTFDRWAEYGGEEWTWDPLVPYLRKSATYHDDTGLYNPELKKLGAGGPIPISHSELVEELEPFRENLIKAWKSTGKPFTENIYDGEMIGLNHCISTIYHGKRSGSFLFVKNRPNITIIPEVHSKNLIIDASNTAKGVVVIDKEGNEHSFYATREVILSQGVFESPKLLMLSGVGPRKELESNGIEVKVESRHVGQNLLDHPGVPFVLQVKDDICVDDILMRQNEKNKAAHVQYQKDGSGPVGSGLLELVGFPRIDEYFEKDPLYRERKAANGGKDPFCPEGQPHFELDFVGMYGTAFQWHFPTPKKGSHITIVVDLVRPVSEGGEVTLNSADPLEQPKINLNFFADELDIVGMREGIRFTYDLLTKGDGFKDLVVKEFPWEMPLDDDKEMRRAVLDRCQTAFHPCGTNRLSKNIEQGVVDPALKVHGVKNLRVIDASIIPVIPDCRIQNSVYMIGEKGADLIKAAHKDLYNLEHHHHHH

SEQ ID NO: 20 - protein, 23 aa, Schwanniomyces vanrijiae
  misc_feature (3)..(3): Xaa can be any naturally occurring amino acid
SHXDFVIVGGGTAGNTVAGRLAE

SEQ ID NO: 21 - protein, 20 aa, Schwanniomyces vanrijiae
DFVIVGGGTAGNTVAGRLAE

SEQ ID NO: 22 - protein, 5 aa, Schwanniomyces vanrijiae
DWAYK

SEQ ID NO: 23 - protein, 16 aa, Schwanniomyces vanrijiae
  misc_feature (4)..(4): Xaa can be any naturally occurring amino acid
  misc_feature (6)..(6): Xaa can be any naturally occurring amino acid
  misc_feature (8)..(8): Xaa can be any naturally occurring amino acid
  misc_feature (11)..(11): Xaa can be any naturally occurring amino acid
TFDXWXEXGGXEWTWD

SEQ ID NO: 24 - protein, 12 aa, Schwanniomyces vanrijiae
TFDWEGGEWTWD

SEQ ID NO: 25 - protein, 12 aa, Schwanniomyces vanrijiae
EWTWDPLVPYLR

SEQ ID NO: 26 - protein, 3 aa, Schwanniomyces vanrijiae
DLY

SEQ ID NO: 27 - protein, 407 aa, Pseudomonas putida
MAGNKSVVYHGTRDLRVETVPYPKLEHNNRKLEHAVILKVVSTNICGSDQHIYRGRFIVPKGHVLGHEITGEVVEKGSDVELMDIGDLVSVPFNVACGRCRNCKEARSDVCENNLVNPDADLGAFGFDLKGWSGGQAEYVLVPYADYMLLKFGDKEQAMEKIKDLTLISDILPTGFHGCVSAGVKPGSHVYIAGAGPVGRCAAAGARLLGAACVIVGDQNPERLKLLSDAGFETIDLRNSAPLRDQIDQILGKPEVDCGVDAVGFEAHGLGDEANTETPNGALNSLFDVVRAGGAIGIPGIYVGSDPDPVNKDAGSGRLHLDFGKMWTKSIRIMTGMAPVTNYNRHLTEAILWDQMPYLSKVMNIEVITLDQAPDGYAKFDKGSPAKFVIDPHGMLKNKLEHHHHHH

SEQ ID NO: 28 - protein, 6 aa, Pseudomonas putida
  misc_feature (2)..(2): Xaa can be any naturally occurring amino acid
  misc_feature (4)..(5): Xaa can be any naturally occurring amino acid
GXGXXG

SEQ ID NO: 29 - protein, 8 aa, Pseudomonas putida
  misc_feature (2)..(2): Xaa can be any naturally occurring amino acid
  misc_feature (4)..(5): Xaa can be any naturally occurring amino acid
  misc_feature (7)..(7): Xaa can be any naturally occurring amino acid
GXGXXGXD

SEQ ID NO: 30 - protein, 9 aa, Pseudomonas putida
  misc_feature (2)..(2): Xaa can be any naturally occurring amino acid
  misc_feature (5)..(6): Xaa can be any naturally occurring amino acid
  misc_feature (8)..(8): Xaa can be any naturally occurring amino acid
GXGGXXGXD

SEQ ID NO: 31 - protein, 9 aa, Pseudomonas putida
  misc_feature (2)..(2): Xaa can be any naturally occurring amino acid
  misc_feature (5)..(6): Xaa can be any naturally occurring amino acid
  misc_feature (8)..(8): Xaa can be any naturally occurring amino acid
GXGGXXGXD

SEQ ID NO: 32 - protein, 18 aa, Pseudomonas putida
  misc_feature (2)..(2): Xaa can be any naturally occurring amino acid
  misc_feature (4)..(4): Xaa can be any naturally occurring amino acid
  misc_feature (7)..(7): Xaa can be any naturally occurring amino acid
  misc_feature (9)..(9): Xaa can be any naturally occurring amino acid
  misc_feature (13)..(14): Xaa can be any naturally occurring amino acid
  misc_feature (16)..(17): Xaa can be any naturally occurring amino acid
GXGXVGXGXGAAXXIXXD

SEQ ID NO: 33 - protein, 8 aa, Pseudomonas putida
  misc_feature (2)..(2): Xaa can be any naturally occurring amino acid
  misc_feature (4)..(5): Xaa can be any naturally occurring amino acid
  misc_feature (7)..(7): Xaa can be any naturally occurring amino acid
CXSXXHXH

SEQ ID NO: 34 - protein, 13 aa, Pseudomonas putida
  misc_feature (2)..(2): Xaa can be any naturally occurring amino acid
  misc_feature (5)..(5): Xaa can be any naturally occurring amino acid
  misc_feature (7)..(7): Xaa can be any naturally occurring amino acid
  misc_feature (9)..(9): Xaa can be any naturally occurring amino acid
  misc_feature (11)..(11): Xaa can be any naturally occurring amino acid
CXSDXHXGXPXGH

SEQ ID NO: 35 - protein, 9 aa, Pseudomonas putida
  misc_feature (2)..(3): Xaa can be any naturally occurring amino acid
  misc_feature (5)..(6): Xaa can be any naturally occurring amino acid
  misc_feature (8)..(8): Xaa can be any naturally occurring amino acid
CXXCXXCXC

SEQ ID NO: 36 - protein, 10 aa, Pseudomonas putida
  misc_feature (3)..(3): Xaa can be any naturally occurring amino acid
  misc_feature (6)..(6): Xaa can be any naturally occurring amino acid
  misc_feature (9)..(9): Xaa can be any naturally occurring amino acid
CGXCRXCKXC

SEQ ID NO: 37 - protein, 1337 aa, Artificial Sequence
  Synthetic
MVQSHYDFVIVGGGTAGNTVAGRLAENPNVTVLVVEAGVANSADLPEITTPSNAMNLRGSKHDWAYKTTLVKRDDYERIEKPNTRGKALGGSSSLNYFTWIPGCKPTFDRWAEYGGEEWTWDPLVPYLRKSATYHDDTGLYNPELKKLGAGGPIPISHSELVEELEPFRENLIKAWKSTGKPFTENIYDGEMIGLNHCISTIYHGKRSGSFLFVKNRPNITIIPEVHSKNLIIDASNTAKGVVVIDKEGNEHSFYATREVILSQGVFESPKLLMLSGVGPRKELESNGIEVKVESRHVGQNLLDHPGVPFVLQVKDDICVDDILMRQNEKNKAAHVQYQKDGSGPVGSGLLELVGFPRIDEYFEKDPLYRERKAANGGKDPFCPEGQPHFELDFVGMYGTAFQWHFPTPKKGSHITIVVDLVRPVSEGGEVTLNSADPLEQPKINLNFFADELDIVGMREGIRFTYDLLTKGDGFKDLVVKEFPWEMPLDDDKEMRRAVLDRCQTAFHPCGTNRLSKNIEQGVVDPALKVHGVKNLRVIDASIIPVIPDCRIQNSVYMIGEKGADLIKAAHKDLYNGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGHMGHPEEVDVIVCGGGPAGCVVAGRLAYADPTLKVMLIEGGANNRDDPWVYRPGIYVRNMQRNGINDKATFYTDTMASSYLRGRRSIVPCANILGGGSSINFQMYTRASASDWDDFKTEGWTCKDLLPLMKRLENYQKPCNNDTHGYDGPIAISNGGQIMPVAQDFLRAAHAIGVPYSDDIQDLTTAHGAEIWAKYINRHTGRRSDAATAYVHSVMDVQDNLFLRCNARVSRVLFDDNNKAVGVAYVPSRNRTHGGKLHETIVKARKMVVLSSGTLGTPQILERSGVGNGELLRQLGIKIVSDLPGVGEQYQDHYTTLSIYRVSNESITTDDFLRGVKDVQRELFTEWEVSPEKARLSSNAIDAGFKIRPTEEELKEMGPEFNELWNRYFKDKPDKPVMFGSIVAGAYADHTLLPPGKYITMFQYLEYPASRGKIHIKSQNPYVEPFFDSGFMNNKADFAPIRWSYKKTREVARRMDAFRGELTSHHPRFHPASPAACKDIDIETAKQIYPDGLTVGIHMGSWHQPSEPYKHDKVIEDIPYTEEDDKAIDDWVADHVETTWHSLGTCAMKPREQGGVVDKRLNVYGTQNLKCVDLSICPDNLGTNTYSSALLVGEKGADLIAEELGLKIKTPHAPVPHAPVPTGRPATQQVR

SEQ ID NO: 38 - protein, 1230 aa, Pseudomonas putida
MVQSHYDFVIVGGGTAGNTVAGRLAENPNVTVLVVEAGVANSADLPEITTPSNAMNLRGSKHDWAYKTTLVKRDDYERIEKPNTRGKALGGSSSLNYFTWIPGCKPTFDRWAEYGGEEWTWDPLVPYLRKSATYHDDTGLYNPELKKLGAGGPIPISHSELVEELEPFRENLIKAWKSTGKPFTENIYDGEMIGLNHCISTIYHGKRSGSFLFVKNRPNITIIPEVHSKNLIIDASNTAKGVVVIDKEGNEHSFYATREVILSQGVFESPKLLMLSGVGPRKELESNGIEVKVESRHVGQNLLDHPGVPFVLQVKDDICVDDILMRQNEKNKAAHVQYQKDGSGPVGSGLLELVGFPRIDEYFEKDPLYRERKAANGGKDPFCPEGQPHFELDFVGMYGTAFQWHFPTPKKGSHITIVVDLVRPVSEGGEVTLNSADPLEQPKINLNFFADELDIVGMREGIRFTYDLLTKGDGFKDLVVKEFPWEMPLDDDKEMRRAVLDRCQTAFHPCGTNRLSKNIEQGVVDPALKVHGVKNLRVIDASIIPVIPDCRIQNSVYMIGEKGADLIKAAHKDLYNGGHMGHPEEVDVIVCGGGPAGCVVAGRLAYADPTLKVMLIEGGANNRDDPWVYRPGIYVRNMQRNGINDKATFYTDTMASSYLRGRRSIVPCANILGGGSSINFQMYTRASASDWDDFKTEGWTCKDLLPLMKRLENYQKPCNNDTHGYDGPIAISNGGQIMPVAQDFLRAAHAIGVPYSDDIQDLTTAHGAEIWAKYINRHTGRRSDAATAYVHSVMDVQDNLFLRCNARVSRVLFDDNNKAVGVAYVPSRNRTHGGKLHETIVKARKMVVLSSGTLGTPQILERSGVGNGELLRQLGIKIVSDLPGVGEQYQDHYTTLSIYRVSNESITTDDFLRGVKDVQRELFTEWEVSPEKARLSSNAIDAGFKIRPTEEELKEMGPEFNELWNRYFKDKPDKPVMFGSIVAGAYADHTLLPPGKYITMFQYLEYPASRGKIHIKSQNPYVEPFFDSGFMNNKADFAPIRWSYKKTREVARRMDAFRGELTSHHPRFHPASPAACKDIDIETAKQIYPDGLTVGIHMGSWHQPSEPYKHDKVIEDIPYTEEDDKAIDDWVADHVETTWHSLGTCAMKPREQGGVVDKRLNVYGTQNLKCVDLSICPDNLGTNTYSSALLVGEKGADLIAEELGLKIKTPHAPVPHAPVPTGRPATQQVR

SEQ ID NO: 39 - protein, 1738 aa, Pseudomonas putida
MVQSHYDFVIVGGGTAGNTVAGRLAENPNVTVLVVEAGVANSADLPEITTPSNAMNLRGSKHDWAYKTTLVKRDDYERIEKPNTRGKALGGSSSLNYFTWIPGCKPTFDRWAEYGGEEWTWDPLVPYLRKSATYHDDTGLYNPELKKLGAGGPIPISHSELVEELEPFRENLIKAWKSTGKPFTENIYDGEMIGLNHCISTIYHGKRSGSFLFVKNRPNITIIPEVHSKNLIIDASNTAKGVVVIDKEGNEHSFYATREVILSQGVFESPKLLMLSGVGPRKELESNGIEVKVESRHVGQNLLDHPGVPFVLQVKDDICVDDILMRQNEKNKAAHVQYQKDGSGPVGSGLLELVGFPRIDEYFEKDPLYRERKAANGGKDPFCPEGQPHFELDFVGMYGTAFQWHFPTPKKGSHITIVVDLVRPVSEGGEVTLNSADPLEQPKINLNFFADELDIVGMREGIRFTYDLLTKGDGFKDLVVKEFPWEMPLDDDKEMRRAVLDRCQTAFHPCGTNRLSKNIEQGVVDPALKVHGVKNLRVIDASIIPVIPDCRIQNSVYMIGEKGADLIKAAHKDLYNGGSAGNKSVVYHGTRDLRVETVPYPKLEHNNRKLEHAVILKVVSTNICGSDQHIYRGRFIVPKGHVLGHEITGEVVEKGSDVELMDIGDLVSVPFNVACGRCRNCKEARSDVCENNLVNPDADLGAFGFDLKGWSGGQAEYVLVPYADYMLLKFGDKEQAMEKIKDLTLISDILPTGFHGCVSAGVKPGSHVYIAGAGPVGRCAAAGARLLGAACVIVGDQNPERLKLLSDAGFETIDLRNSAPLRDQIDQILGKPEVDCGVDAVGFEAHGLGDEANTETPNGALNSLFDVVRAGGAIGIPGIYVGSDPDPVNKDAGSGRLHLDFGKMWTKSIRIMTGMAPVTNYNRHLTEAILWDQMPYLSKVMNIEVITLDQAPDGYAKFDKGSPAKFVIDPHGMLKNKGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGHMGHPEEVDVIVCGGGPAGCVVAGRLAYADPTLKVMLIEGGANNRDDPWVYRPGIYVRNMQRNGINDKATFYTDTMASSYLRGRRSIVPCANILGGGSSINFQMYTRASASDWDDFKTEGWTCKDLLPLMKRLENYQKPCNNDTHGYDGPIAISNGGQIMPVAQDFLRAAHAIGVPYSDDIQDLTTAHGAEIWAKYINRHTGRRSDAATAYVHSVMDVQDNLFLRCNARVSRVLFDDNNKAVGVAYVPSRNRTHGGKLHETIVKARKMVVLSSGTLGTPQILERSGVGNGELLRQLGIKIVSDLPGVGEQYQDHYTTLSIYRVSNESITTDDFLRGVKDVQRELFTEWEVSPEKARLSSNAIDAGFKIRPTEEELKEMGPEFNELWNRYFKDKPDKPVMFGSIVAGAYADHTLLPPGKYITMFQYLEYPASRGKIHIKSQNPYVEPFFDSGFMNNKADFAPIRWSYKKTREVARRMDAFRGELTSHHPRFHPASPAACKDIDIETAKQIYPDGLTVGIHMGSWHQPSEPYKHDKVIEDIPYTEEDDKAIDDWVADHVETTWHSLGTCAMKPREQGGVVDKRLNVYGTQNLKCVDLSICPDNLGTNTYSSALLVGEKGADLIAEELGLKIKTPHAPVPHAPVPTGRPATQQVR

SEQ ID NO: 40 - protein, 110 aa, Artificial Sequence
  Synthetic
GSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGH

SEQ ID NO: 41 - protein, 511 aa, Artificial Sequence
  Synthetic
GGSAGNKSVVYHGTRDLRVETVPYPKLEHNNRKLEHAVILKVVSTNICGSDQHIYRGRFIVPKGHVLGHEITGEVVEKGSDVELMDIGDLVSVPFNVACGRCRNCKEARSDVCENNLVNPDADLGAFGFDLKGWSGGQAEYVLVPYADYMLLKFGDKEQAMEKIKDLTLISDILPTGFHGCVSAGVKPGSHVYIAGAGPVGRCAAAGARLLGAACVIVGDQNPERLKLLSDAGFETIDLRNSAPLRDQIDQILGKPEVDCGVDAVGFEAHGLGDEANTETPNGALNSLFDVVRAGGAIGIPGIYVGSDPDPVNKDAGSGRLHLDFGKMWTKSIRIMTGMAPVTNYNRHLTEAILWDQMPYLSKVMNIEVITLDQAPDGYAKFDKGSPAKFVIDPHGMLKNKGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGH

SEQ ID NO: 42 - protein, 13 aa, Artificial Sequence
  Synthetic
MGSSHHHHHHGGS

(19) United States
(12) Patent Application Publication
(10) Pub. No.: US 2024/0093245 A1
(43) Pub. Date: Mar. 21, 2024

(54) Compositions and methods for converting methanol into hydrogen peroxide and carbon dioxide

(71) Applicant: The Regents of the University of California, Oakland, CA (US)

(72) Inventors: Christopher B. Eiben, Berkeley, CA (US); Jay D. Keasling, Berkeley, CA (US)

(73) Assignee: The Regents of the University of California, Oakland, CA (US)

(21) Appl. No.: 17/717,141

(22) Filed: Apr. 11, 2022

Related U.S. Application Data
(63) Continuation of application No. PCT/US20/54903, filed on Oct. 9, 2020.
(60) Provisional application No. 62/913,627, filed on Oct. 10, 2019.

(51) Int. Cl. C12P 3/00; C12N 9/02; C12N 9/04; C12N 9/08

(52) U.S. Cl. CPC C12P 3/00; C12N 9/0006; C12N 9/0008; C12N 9/0065; C07K 2319/00; C12Y 101/03013; C12Y 102/03001; C12Y 102/99004; C12Y 111/01021

(57) ABSTRACT

The present invention provides for a method for producing hydrogen peroxide comprising: (a) contacting a methanol with a methanol oxidase bound with a flavin adenine dinucleotide (FAD) cofactor, such that the methanol is oxidized into a formaldehyde and the FAD cofactor is reduced into FADH₂; (b) contacting the formaldehyde with the methanol oxidase or a formate oxidase bound with a FAD cofactor, such that the formaldehyde is oxidized into a formate and the FAD is reduced into FADH₂; and (c) contacting oxygen with one or more of the FADH₂ to produce hydrogen peroxide and oxidize FADH₂ into FAD. The present invention also provides for a fusion protein comprising any two or all of methanol oxidase, formate oxidase, and formaldehyde dismutase.

The invention was made with government support under Contract Nos. DE-AC02-05CH11231 awarded by the U.S. Department of Energy. The government has certain rights in the invention.

Reference to a Sequence Listing A Sequence Listing in text format is incorporated by reference into the specification. The name of the text file containing the Sequence Listing is SeqList_ST25B.txt. The text file is 83,456 bytesbytes and was created and submitted electronically via EFS-Web on Nov. 29, 2023.

### BACKGROUND OF THE INVENTION

Hydrogen peroxide is a commodity chemical produced at 4.5 billion kilos per year via the anthraquinone process. The current process for producing hydrogen peroxide is the anthraquinone process, which requires large capital-intensive facilities in order to produce hydrogen peroxide economically. This means customers must ship hydrogen peroxide long distances and store it on site, which is expensive and potentially dangerous due to its chemical instability. What is needed is an alternate process to make hydrogen peroxide that should enable hydrogen peroxide production on site with less capital-intensive equipment.

### SUMMARY OF THE INVENTION

The present invention provides for a method for producing hydrogen peroxide comprising: (a) contacting a methanol with a methanol oxidase bound with a flavin adenine dinucleotide (FAD) cofactor, such that the methanol is oxidized into a formaldehyde and the FAD cofactor is reduced into FADH₂; (b) contacting the formaldehyde with the methanol oxidase or a formate oxidase bound with a FAD cofactor, such that the formaldehyde is oxidized into a formate and the FAD is reduced into FADH₂; and (c) contacting oxygen with one or more of the FADH₂ to produce hydrogen peroxide and oxidize FADH₂ into FAD; or (a) contacting a methanol with a fusion protein of the present invention comprising (i) a methanol oxidase bound with a flavin adenine dinucleotide (FAD) cofactor, and (ii) a formate oxidase bound with a FAD cofactor, such that the methanol oxidase oxidizes the methanol into a formaldehyde and the FAD cofactor bound to the methanol oxidase is reduced into FADH₂; (b) contacting the formaldehyde with the methanol oxidase or the formate oxidase, such that the formaldehyde is oxidized into a formate and the FAD of an unreduced methanol oxidase or formate oxidase is reduced into FADH₂; and (c) contacting oxygen with one or more of the FADH₂ to produce hydrogen peroxide and oxidize FADH₂ into FAD. In preferred variations, methanol oxidase and formate oxidase are enzymes extracted from distinct organisms (e.g., methanol oxidase is extracted from Phanerochaete chryosporeium and formate oxidase is extracted from Schwanniomyces vanrijiae) or are functional variants of the organism enzymes (e.g. methanol oxidase from Phanerochaete chryosporeium expressed in Escherichia coli recombinantly and/or formate oxidase from Schwanniomyces vanrijiae expressed in Escherichia coli recombinantly).

The present invention provides for a fusion protein comprising any two or all of methanol oxidase, formate oxidase, and formaldehyde dismutase, wherein each enzyme is linked via a linker to another enzyme. In preferred variations, methanol oxidase, formate oxidase, and/or formaldehyde dismutase are enzymes extracted from distinct organisms (e.g., methanol oxidase is extracted from Phanerochaete chryosporeium, formate oxidase is extracted from Schwanniomyces vanrijiae, and/or formaldehyde dismutase is extracted from Pseudomonas putida) or are functional variants of the organism extracted enzymes.

In some embodiments, the method for producing hydrogen peroxide comprises: (a) contacting a methanol with a fusion protein comprising a methanol oxidase bound with a flavin adenine dinucleotide (FAD) cofactor, such that the methanol is oxidized into a formaldehyde and the FAD cofactor is reduced into FADH₂; (b) contacting the formaldehyde with the methanol oxidase or a formate oxidase bound with a FAD cofactor, such that the formaldehyde is oxidized into a formate and the FAD is reduced into FADH₂; and (c) contacting oxygen with one or more of the FADH₂ to produce hydrogen peroxide and oxidize FADH₂ into FAD.

The present invention provides for an in vitro composition comprising a methanol oxidase and a formate oxidase, or a fusion protein of the present invention, or a mixture thereof.

The present invention provides for a host cell comprising a polynucleotide encoding a methanol oxidase, formate oxidase, and/or formaldehyde dismutase, and/or a fusion protein of the present invention, each operatively linked to separate promoters or one promoter.

The present invention provides for a method of producing a methanol oxidase, formate oxidase, and/or formaldehyde dismutase comprising: (a) providing a host cell of the present invention in a nutrient medium, and (b) culturing or growing the host cell in the nutrient medium such that the methanol oxidase, formate oxidase, and/or formaldehyde dismutase is expressed or produced.

The present invention may be implemented as a method for producing hydrogen peroxide from a methanol source as part of an at least three step reaction, wherein the method leverages enzymatic properties of a methanol oxidase, a formaldehyde dismutase, and/or a formate oxidase to complete the reactions, and wherein the steps comprise: (a) oxidizing methanol to a formaldehyde by reducing FAD to FADH₂, (b) oxidizing the formaldehyde to a formate by reducing FAD to an FADH₂, and (c) reducing an O₂ to hydrogen peroxide by oxidizing the FADH₂ to an FAD. In this manner, the method may be implemented for general hydrogen peroxide production (e.g. as an industrial chemical product). Additionally, the method may be implemented as part of a paper production process.

The method may be alternatively implemented as a one step, or multiple steps, of the methanol to hydrogen peroxide process. In a first implementation, oxidizing methanol to a formaldehyde while reducing FAD to FADH₂, using either methanol oxidase and/or formate oxidase, may be implemented as a standalone process. This implementation may be useful for the production of formaldehyde and/or the removal/breakdown of methanol.

In a second implementation: oxidizing formaldehyde to formate while reducing FAD to FADH₂, using methanol oxidase and/or formate oxidase, may be implemented as a standalone process. This second implementation may be useful for the production of formate.

In a third implementation: converting oxygen into hydrogen peroxide in conjunction with oxydizing FADH₂ into FAD, using the reduced form (i.e. bound to FADH₂) of methanol oxidase and/or formate oxidase, may be implemented as a standalone process. This third implementation may be useful for the production of hydrogen peroxide from available FADH₂.

In a fourth implementation: converting formate into carbon dioxide while reducing FAD to FADH₂, using methanol oxidase and/or formate oxidase, may be implemented as a standalone process. This fourth implementation may be used for formate decontamination.

In a fifth implementation, comprising a multi-step process: converting formaldehyde to hydrogen peroxide, the method may be implemented for formaldehyde decontamination.

The foregoing aspects and others will be readily appreciated by the skilled artisan from the following description of illustrative embodiments when read in conjunction with the accompanying drawings.

### BRIEF DESCRIPTION OF THE DRAWINGS

FIG. 1. Comparison of the amino acid sequences of Phanerochaete chrysosporium methanol oxidase (SEQ ID NO:1) and Komagataella phaffii strain GS115 alcohol oxidase (SEQ ID NO:37).

FIG. 2. Putative NAD⁺-binding domain of formaldehyde dismutase and sequence similarity of nucleotide-binding domains. The conserved glycine residues found in the nucleotide-binding domains are outlined in black. The conserved aspartic acid residue is shown below the asterisk. The conserved hydrophobic amino acid residues are below the dots. A “-” represents gaps in the sequences made for alignment of amino acids. 1, Formaldehyde dismutase from P. putida F61 (188-218 of amino acid numbers) (SEQ ID NO:3); 2, alcohol dehydrogenase from horse liver (194-224) (SEQ ID NO:38); 3, lactate dehydrogenase from dogfish muscle (22-53) (SEQ ID NO:39); 4, glyceraldehyde-3-phosphate dehydrogenase from lobster (2-23) (SEQ ID NO:40). (Figure from: Yanase et al., Biosci. Biotech. Biochem, 59:197-202, 1995.)

FIG. 3. Comparison of the amino acid sequence of the P. putida formaldehyde dismutase (SEQ ID NO:3) and E subunit of alcohol dehydrogenase from horse liver (SEQ ID NO:38). The putative ligands of a catalytic Mg²⁺ or Zn²⁺ atom and/or a second Mg²⁺ or Zn²⁺ atom are indicated by closed and open triangles, respectively. The predicted NAD⁺-binding domain is enclosed in parallel lines. (Figure from: Yanase et al., Biosci. Biotech. Biochem, 59:197-202, 1995.)

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

The term “functional variant” refers to a protein, such as an enzyme or transcription factor, that has an amino acid sequence that is at least 70%, 75%, 80%, 85%, 90%, 95% or 99% identical to the amino acid sequence of any one of the proteins described in this specification or in an incorporated reference. The functional variant retains amino acids residues that are recognized as conserved for the protein. The functional variant may have non-conserved amino acid residues replaced or found to be of a different amino acid, or amino acid(s) inserted or deleted, but which does not affect or has insignificant effect on the enzymatic activity of the functional variant. The functional variant has an enzymatic or biological activity that is identical or essentially identical to the enzymatic or biological activity any one of the proteins described in this specification or in an incorporated reference. The functional variant may be found in nature or be an engineered mutant thereof. The mutant may have one or more amino acids substituted, deleted or inserted, or a combination thereof, as compared to the protein described in this specification or in an incorporated reference. The term “functional variant” can also refer to a nucleotide sequence, such as a promoter, that has a nucleotide sequence that is at least 70%, 75%, 80%, 85%, 90%, 95% or 99% identical to the nucleotide sequence of any one of the nucleotide sequence, such as a promoter, described in this specification or in an incorporated reference.

As used herein, the term “promoter” refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon.

A polynucleotide or amino acid sequence is “heterologous” to an organism or a second polynucleotide or amino acid sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety, or a gene that is not naturally expressed in the target tissue).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “host cell” and “host microorganism” are used interchangeably herein to refer to a living biological cell, such as a microbe, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The present invention provides for a process to make hydrogen peroxide that enables hydrogen peroxide production on site with less capital-intensive equipment. In addition, this process uses a cheaper feedstock than the anthraquinone process which should make this process cost competitive at scale as well. The process uses a two enzyme pathway (comprising methanol oxidase and formate oxidase) to convert one methanol molecule into one carbon dioxide (CO₂) molecule and result producing up to three hydrogen peroxide molecules.

The present invention alternatively provides a process for waste removal, wherein the process enables the removal/breakdown of methanol, formaldehyde and/or formic acid. A process that may be beneficial for waste management, particularly for water treatment. This process may use a one, two, or three enzyme pathway, wherein formaldehyde and/or formate are oxidized along a pathway to convert formaldehyde (or formate) into carbon dioxide, thereby producing hydrogen peroxide as one end product. This proces may additionally include catalases (e.g. KatE or KatG) and/or peroxidases to further break down the hydrogen peroxide.

The present invention provides for a method for producing hydrogen peroxide comprising: (a) contacting a methanol with a methanol oxidase bound with a flavin adenine dinucleotide (FAD) cofactor, such that the methanol is oxidized into a formaldehyde and the FAD cofactor is reduced into FADH₂; (b) contacting the formaldehyde with the methanol oxidase or a formate oxidase bound with a FAD cofactor, such that the formaldehyde is oxidized into a formate and the FAD is reduced into FADH₂; and (c) contacting oxygen with one or more of the FADH₂ to produce hydrogen peroxide and oxidize FADH₂ into FAD. In some embodiments, the method further comprises: (d) contacting the formaldehyde with a formaldehyde dismutase to convert formaldehyde into methanol and formate. In preferred variations, methanol oxidase and formate oxidase are enzymes extracted from distinct organisms (e.g., methanol oxidase is extracted from Phanerochaete chryosporeium and formate oxidase is extracted from Schwanniomyces vanrijiae) or are functional variants of the organism extracted enzymes.

The method may also function as a waste management process. That is, this enzyme pathway may also be used to detoxify methanol, formaldehyde, and/or formate, into CO2 and water. In these variations, the method may further include the addition of catalase enzyme(s) and/or peroxidase(s) enzyme. In the case of catalase, the enzyme net reaction is to convert two H₂O₂ into two H₂O and one O₂ end products. In the case of the peroxidase enzyme, the method first reduces H₂O₂ to water, producing an oxidized peroxidase active site. The peroxidase may then oxidize an organic molecule, including, but not limited to, methanol, formaldehyde, or formate, in order to regenerate the peroxidase active site to its original state. In some variations the method may additionally or alternatively include a hydroperoxidase, wherein hydroperoxidase is a catalase enzyme that further has peroxidase activity. Catalase enzymes may use heme-based cofactors, or alternatively manganese in their active site. In some variations the catalase enzyme may comprise Escherichia coli native catalases (e.g. KatE or KatG). Additionally or alternatively, other catalase or peroxidase enzymes may be implemented as desired.

As the process herein may be implemented at different conditions (e.g. different pH), formaldehyde and hydrated formaldehyde (also referred to as: methanediol, formaldehyde monohydrate, or methylene glycol with the chemical formula CH₂(OH)₂), are considered as functional variants. Unless stated otherwise, any reference to formaldehyde may equally refer to hydrated formaldehyde. Additionally, in the same manner, formate may equally refer to formate or formic acid.

The present invention provides for an in vitro composition comprising a methanol oxidase and a formate oxidase. In some embodiments, the composition is a solution suitable for the methanol oxidase and the formate oxidase (and optionally formaldehyde dismutase) to catalyze their respective enzymatic reactions. In some embodiments, the solution comprises a suitable salt buffer and has a pH having a value from about 4.0, 5.0, 6.0, or 7.0 to about 7.2, 7.3, 7.4, 7.5, 7.6, 7.7, 7.8, 7.9, or 8.0. In some embodiments, the suitable salt buffer is a sodium phosphate, sodium pyrophosphate, or sodium metasilicate buffer. In some embodiments, the suitable salt buffer has a concentration of about 1 μM, 10 μM, 100 μM, or 1 mM, 10 mM, 20 mM, 30 mM, 40 mM, 50 mM, 60 mM, 70 mM, 80 mM, 90 mM, 100 mM, 200 mM, 300 mM, 400 mM, or 500 mM. In some embodiments, the suitable salt buffer has a concentration of at least about 1 μM, 10 μM, 100 μM, 1 mM, 10 mM, 20 mM, 30 mM, 40 mM, or 50 mM. In some embodiments, the suitable salt buffer has a concentration up to about 50 mM, 60 mM, 70 mM, 80 mM, 90 mM, 100 mM, 200 mM, 300 mM, 400 mM, or 500 mM. In some embodiments, the suitable salt buffer is an about 50 mM sodium phosphate buffer. In some embodiments, the solution comprises a suitable salt buffer and has a pH having a value from about 6.0 to about 8.0. In some embodiments, the solution comprises a suitable salt buffer and has a pH having a value from about 6.6 to about 7.8. In some embodiments, the solution comprises a suitable salt buffer and has a pH having a value from about 6.8 to about 7.6. In some embodiments, the solution comprises a suitable salt buffer and has a pH having a value from about 7.0 to about 7.4. In some embodiments, the pH is about 7.2. In some embodiments, the composition further comprises FAD cofactor. In some embodiments, the composition further comprises methanol and oxygen. In some embodiments, the composition further comprises: a formaldehyde dismutase. In some embodiments, the methanol oxidase, formate oxidase, and/or formaldehyde dismutase are isolated or purified.

In some embodiments, the methanol oxidase comprises the amino acid sequence of Phanerochaete chrysosporium methanol oxidase, or a functional variant thereof. In one variation, the methanol oxidase matches the amino acid sequence of Phanerochaete chrysosporium methanol oxidase by at least 90%. In a second variation, the methanol oxidase matches the amino acid sequence of Phanerochaete chrysosporium methanol oxidase by at least 80%. In a third variation, the methanol oxidase matches the amino acid sequence of Phanerochaete chrysosporium methanol oxidase by at least 70%. Depedent on implementation, the methanol oxidase may be purified and extracted from a microorganism (e.g. from Phanerochaete chrysosporium or from an organism with a methanol oxidase that is a functional variant), the methanol oxidase may be synthetically engineered, and/or the methanol oxidase may be produced (e.g. the amino acid sequence is expressed recombinantly in another organism, such as Escherichia coli). The methanol oxidase may, additionally or alternatively, be produced using any other applicable method. Examples of functional variant amino sequences may include, but are not limited to: synthetic variants, the amino acid sequence for the hypothetical protein PHACADRAFT_252324 from Phanerochaete, the amino acid sequence for the hypothetical protein PHLGIDRAFT 120749 from Phlebiopsis gigantea, the amino acid sequence for GMC oxidoreductase from Trametes coccinea, the amino acid sequence for alcohol oxidase from Obba rivulosa, the amino acid sequence for alcohol oxidase from Gelatoporia subvermispora, the hypothetical protein EIP91_001657 from Steccherimum ochraceum, the amino acid sequence for the hypothetical protein EUX98_g4623 from Antrodiella citrinella, the amino acid sequence for alcohol oxidase from Gloeophyllum trabeum ATCC 11539, and the amino acid sequence for the hypothetical protein EW026_g1138 from Phlebia centrifuga.

In some embodiments, the formate oxidase comprises the amino acid sequence of Schwanniomyces vanrijiae formate oxidase, or a functional variant thereof. In one variation, the formate oxidase matches the amino acid sequence of Schwanniomyces vanrijiae formate oxidase by at least 90%. In a second variation, the formate oxidase matches the amino acid sequence of Schwanniomyces vanrijiae formate oxidase by at least 80%. In a third variation, the formate oxidase matches the amino acid sequence of Schwanniomyces vanrijiae formate oxidase by at least 70%. Depedent on implementation, the formate oxidase may be purified and extracted from a microorganism (e.g. from Schwanniomyces vanrijiae or from an organism with a formate oxidase that is a functional variant), the formate oxidase may be synthetically engineered, and/or the formate oxidase may be produced (e.g. the amino acid sequence is expressed recombinantly in another organism, such as Escherichia coli). The formate oxidase may, additionally or alternatively, be produced using any other applicable method. Examples of functional variant amino acid sequences may include, but are not limited to: synthetic variants, the amino acid sequence for a choline dehydrogenase from Zygosaccharomyces baiii, the amino acid sequence for GMC oxidoreductase from Hyaloscypha variabilis, the amino acid sequence for the hypothetical protein B7463_g2234 from Scytalidium lignicola, the amino acid sequence for LAFE_OF18206g1_1 from Lachancea fermentati, the amino acid sequence for the hypothetical protein TDEL_0B00110 from Torulaspora delbrueckii, the amino acid sequence for the hypothetical protein FDECE_1716 from Fusarium decemcellulare, the amino acid sequence for the hypothetical protein CHU98_g3475 from Xylaria longipes, the amino acid sequence for the uncharacterized protein NECHADRAFT_85374 from Fusarium vanettenii, the amino acid sequence for the hypothetical protein PV04_09157 from Phialophora americana, the amino acid sequence for the hypothetical protein PV07_09250 from Cladophialophora immunda, the amino acid sequence for the hypothetical protein ABW21_db0200298 from Drechslerella brochopaga, the amino acid sequence for the hypothetical protein ABW19_dt0209074 from Dactylella cylindrospora, the amino acid sequence for a glucose-methanol-choline oxidoreductase from Aspergillus bombycis, the amino acid sequence for the hypothetical protein BDV34DRAFT_235406 from Aspergillus parasiticus, the amino acid sequence for a glucose-methanol-choline oxidoreductase from Aspergillus nomiae, the amino acid sequence of a predicted protein from Byssochlamys spectabilis, the amino acid sequence for the hypothetical protein CEP54_015966 from Fusarium sp., and the amino acid sequence for the uncharacterized protein BDV37DRAFT_284707 from Aspergillus pseudonomius.

In some embodiments, the formaldehyde dismutase comprises the amino acid sequence of Pseudomonas putida formaldehyde dismutase, or functional variant thereof. In one variation, the formaldehyde dismutase matches the amino acid sequence of Pseudomonas putida formaldehyde dismutase by at least 90%. In a second variation, the formaldehyde dismutase matches the amino acid sequence of Pseudomonas putida formaldehyde dismutase by at least 80%. In a third variation, the formaldehyde dismutase matches the amino acid sequence of Pseudomonas putida formaldehyde dismutase by at least 70%. Depedent on implementation, the formaldehyde dismutase may be purified and extracted from a microorganism (e.g. from Pseudomonas putida or from an organism with a formaldehyde dismutase that is a functional variant), the formaldehyde dismutase may be synthetically engineered, and/or the formaldehyde dismutase may be produced (e.g. the amino acid sequence is expressed recombinantly in another organism, such as Escherichia coli). The formaldehyde dismutase may, additionally or alternatively, be produced using any other applicable method. Examples of functional variant amino acid sequences may include, but are not limited to: synthetic variants, the amino acid sequence for the alcohol dehydrogenase catalytic domain-containing protein from Pseudomonas monteiii, the amino acid sequence for the alcohol dehydrogenase catalytic domain-containing protein from Phaeobacter piscinae, and the amino acid sequence for the aldehyde dehydrogenase from Sinorhizobium sp.

Oxygen regenerates the FAD cofactor and is converted into hydrogen peroxide through the following reaction:

O₂+FADH₂→H₂O₂+FAD

Formaldehyde dismutase catalyzes the following reaction:

2HCHO+H₂O→H₃COH+HCOOH

To convert methanol to hydrogen peroxide and CO₂, two enzymes, comprising methanol oxidase and formate oxidase, are used. Methanol oxidase uses a tightly bound FAD cofactor to oxidize methanol to formladehyde. Oxygen regenerates the cofactor to FAD and releases hydrogen peroxide in the process. Both methanol oxidase and formate oxidase can convert formaldehyde to formate by the same process as before via a tightly bound FAD cofactor yielding a hydrogen peroxide. Formate oxidase then converts formate into CO₂ and releases a hydrogen peroxide in the process. A third enzyme, formaldehyde dismutase, can be added to the process which may be advantageous at high substrate concentrations. Formaldehyde dismutase converts two formaldehyde and one water into one methanol and one formate. The terms formate and the conjugate base, formic acid are used interchangeably herein. Since methanol oxidase and formate oxidase did not evolve specifically to use formaldehyde as a substrate, formaldehyde dismutase may help keep a low formaldehyde concentration without hurting the stoichiometry of the process.

The amino acid sequence of Phanerochaete chrysosporium methanol oxidase comprises the following:

(SEQ ID NO: 1)
MGHPEEVDVIVCGGGPAGCVVAGRLAYADPTLKVMLIEGG
ANNRDDPWVYRPGIYVRNMQRNGINDKATFYTDTMASSYL
RGRRSIVPCANILGGGSSINFQMYTRASASDWDDFKTEGW
TCKDLLPLMKRLENYQKPCNNDTHGYDGPIAISNGGQIMP
VAQDFLRAAHAIGVPYSDDIQDLTTAHGAEIWAKYINRHT
GRRSDAATAYVHSVMDVQDNLFLRCNARVSRVLFDDNNKA
VGVAYVPSRNRTHGGKLHETIVKARKMVVLSSGTLGTPQI
LERSGVGNGELLRQLGIKIVSDLPGVGEQYQDHYTTLSIY
RVSNESITTDDFLRGVKDVQRELFTEWEVSPEKARLSSNA
IDAGFKIRPTEEELKEMGPEFNELWNRYFKDKPDKPVMFG
SIVAGAYADHILLPPGKYITMFQYLEYPASRGKIHIKSQN
PYVEPFFDSGFMNNKADFAPIRWSYKKTREVARRMDAFRG
ELTSHHPRFHPASPAACKDIDIETAKQIYPDGLTVGIHMG
SWHQPSEPYKHDKVIEDIPYTEEDDKAIDDWVADHVETTW
HSLGTCAMKPREQGGVVDKRLNVYGTQNLKCVDLSICPDN
LGTNTYSSALLVGEKGADLIAEELGLKIKTPHAPVPHAPV
PTGRPATQQVR

The amino acid sequence of the enzyme expressed by pHP30 is as follows:

(SEQ ID NO: 4)
MGSSHHHHHHGSGLVPRGSASMSDSEVNQEAKPEVKPEVK
PETHINLKVSDGSSEIFFKIKKTTPLRRLMEAFAKRQGKE
MDSLRFLYDGIRIQADQTPEDLDMEDNDIIEAHREQIGGH
MGHPEEVDVIVCGGGPAGCVVAGRLAYADPTLKVMLIEGG
ANNRDDPWVYRPGIYVRNMQRNGINDKATFYTDTMASSYL
RGRRSIVPCANILGGGSSINFQMYTRASASDWDDFKTEGW
TCKDLLPLMKRLENYQKPCNNDTHGYDGPIAISNGGQIMP
VAQDFLRAAHAIGVPYSDDIQDLITAHGAEIWAKYINRHT
GRRSDAATAYVHSVMDVQDNLFLRCNARVSRVLFDDNNKA
VGVAYVPSRNRTHGGKLHETIVKARKMVVLSSGTLGTPQI
LERSGVGNGELLRQLGIKIVSDLPGVGEQYQDHYTTLSIY
RVSNESITTDDFLRGVKDVQRELFTEWEVSPEKARLSSNA
IDAGFKIRPTEEELKEMGPEFNELWNRYFKDKPDKPVMFG
SIVAGAYADHTLLPPGKYITMFQYLEYPASRGKIHIKSQN
PYVEPFFDSGFMNNKADFAPIRWSYKKTREVARRMDAFRG
ELTSHHPRFHPASPAACKDIDIETAKQIYPDGLTVGIHMG
SWHQPSEPYKHDKVIEDIPYTEEDDKAIDDWVADHVETTW
HSLGTCAMKPREQGGVVDKRLNVYGTQNLKCVDLSICPDN
LGTNTYSSALLVGEKGADLIAEELGLKIKTPHAPVPHAPV
PTGRPATQQVR

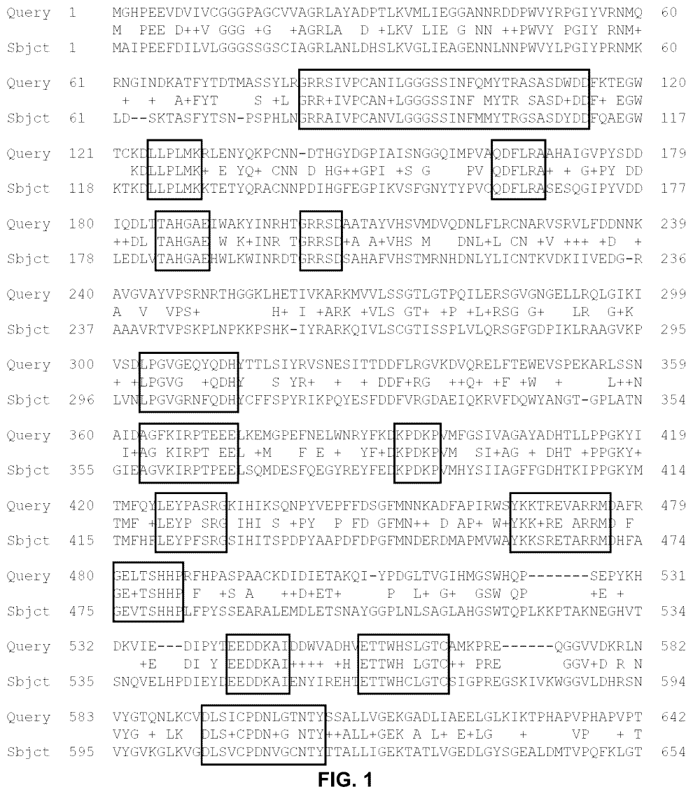

In some embodiments, the methanol oxidase has an amino acid sequence having at least 70% amino acid residue identity with SEQ ID NO:1. In some embodiments, the methanol oxidase comprises at last one or more, or all, of the following conserved sequences: GRRXIVPCANILGGGSSINFXMYTRXSASDXDD (SEQ ID NO:5), LLPLXK (SEQ ID NO:6), QDFLRA (SEQ ID NO:7), TAHGAE (SEQ ID NO:8), GRRSD (SEQ ID NO:9), LPGVGXXXQDH (SEQ ID NO:10), AGXKIRPTXEE (SEQ ID NO:11), KPDKP (SEQ ID NO:12), LEYPXSRG (SEQ ID NO:13), YKKXREXARRM (SEQ ID NO:14), GEXTSHHP (SEQ ID NO:15), EEDDXAI (SEQ ID NO:16), ETTWHXLGTC (SEQ ID NO:17), and DLSXCPDNXGXNTY (SEQ ID NO:18); wherein “X” is any amino acid residue. Certain of these conserved sequences are disclosed in FIG. 1.

The amino acid sequence of Schwanniomyces vanrijiae formate oxidase comprises the following:

(SEQ ID NO: 2)
MVQSHYDFVIVGGGTAGNTVAGRLAENPNVTVLVVEAGVA
NSADLPEITTPSNAMNLRGSKHDWAYKTTLVKRDDYERIE
KPNTRGKALGGSSSLNYFTWIPGCKPTFDRWAEYGGEEWT
WDPLVPYLRKSATYHDDTGLYNPELKKLGAGGPIPISHSE
LVEELEPFRENLIKAWKSTGKPFTENIYDGEMIGLNHCIS
TIYHGKRSGSFLFVKNRPNITIIPEVHSKNLIIDASNTAK
GVVVIDKEGNEHSFYATREVILSQGVFESPKLLMLSGVGP
RKELESNGIEVKVESRHVGQNLLDHPGVPFVLQVKDDICV
DDILMRQNEKNKAAHVQYQKDGSGPVGSGLLELVGFPRID
EYFEKDPLYRERKAANGGKDPFCPEGQPHFELDFVGMYGT
AFQWHFPTPKKGSHITIVVDLVRPVSEGGEVTLNSADPLE
QPKINLNEFADELDIVGMREGIRFTYDLLTKGDGFKDLVV
KEFPWEMPLDDDKEMRRAVLDRCQTAFHPCGTNRLSKNIE
QGVVDPALKVHGVKNLRVIDASIIPVIPDCRIQNSVYMIG
EKGADLIKAAHKDLYN

The amino acid sequence of the enzyme expressed by pHP2 is as follows:

(SEQ ID NO: 19)
MVQSHYDFVIVGGGTAGNTVAGRLAENPNVTVLVVEAGVA
NSADLPEITTPSNAMNLRGSKHDWAYKTTLVKRDDYERIE
KPNTRGKALGGSSSLNYFTWIPGCKPTFDRWAEYGGEEWT
WDPLVPYLRKSATYHDDTGLYNPELKKLGAGGPIPISHSE
LVEELEPFRENLIKAWKSTGKPFTENIYDGEMIGLNHCIS
TIYHGKRSGSFLFVKNRPNITIIPEVHSKNLIIDASNTAK
GVVVIDKEGNEHSFYATREVILSQGVFESPKLLMLSGVGP
RKELESNGIEVKVESRHVGQNLLDHPGVPFVLQVKDDICV
DDILMRQNEKNKAAHVQYQKDGSGPVGSGLLELVGFPRID
EYFEKDPLYRERKAANGGKDPFCPEGQPHFELDFVGMYGT
AFQWHFPTPKKGSHITIVVDLVRPVSEGGEVTLNSADPLE
QPKINLNFFADELDIVGMREGIRFTYDLLTKGDGFKDLVV
KEFPWEMPLDDDKEMRRAVLDRCQTAFHPCGTNRLSKNIE
QGVVDPALKVHGVKNLRVIDASIIPVIPDCRIQNSVYMIG
EKGADLIKAAHKDLYNLEHHHHHH

In some embodiments, the formate oxidase has an amino acid sequence having at least 70% amino acid residue identity with SEQ ID NO:2. In some embodiments, the formate oxidase comprises at last one or more, or all, of the following conserved sequences: SHXDFVIVGGGTAGNTVAGRLAE (SEQ ID NO:20), SH(Y or F)DFVIVGGGTAGNTVAGRLAE (SEQ ID NO:21), DWAYK (SEQ ID NO:22), TFDXWXEXGGXEWTWD (SEQ ID NO:23), TFD(R or Q)W(A or E)E(Y or F)GG(E or K)EWTWD (SEQ ID NO:24), EWTWDPLVPYLR (SEQ ID NO:25), and DLY (SEQ ID NO:26); wherein “X” is any amino acid residue. Certain of these conserved sequences are disclosed in Maeda et al., Biosci. Biotech. Biochem, 72:1999-2004, 2008.

The amino acid sequence of Pseudomonas putida formaldehyde dismutase comprises the following:

(SEQ ID NO: 3)
MAGNKSVVYHGTRDLRVETVPYPKLEHNNRKLEHAVILKV
VSTNICGSDQHIYRGRFIVPKGHVLGHEITGEVVEKGSDV
ELMDIGDLVSVPFNVACGRCRNCKEARSDVCENNLVNPDA
DLGAFGFDLKGWSGGQAEYVLVPYADYMLLKFGDKEQAME
KIKDLTLISDILPTGFHGCVSAGVKPGSHVYIAGAGPVGR
CAAAGARLLGAACVIVGDQNPERLKLLSDAGFETIDLRNS
APLRDQIDQILGKPEVDCGVDAVGFEAHGLGDEANTETPN
GALNSLFDVVRAGGAIGIPGIYVGSDPDPVNKDAGSGRLH
LDFGKMWTKSIRIMTGMAPVINYNRHLTEAILWDQMPYLS
KVMNIEVITLDQAPDGYAKFDKGSPAKFVIDPHGMLKNKL

The amino acid sequence of the enzyme expressed by pHP23 is as follows:

(SEQ ID NO: 27)
MAGNKSVVYHGTRDLRVETVPYPKLEHNNRKLEHAVILKV
VSTNICGSDQHIYRGRFIVPKGHVLGHEITGEVVEKGSDV
ELMDIGDLVSVPENVACGRCRNCKEARSDVCENNLVNPDA
DLGAFGFDLKGWSGGQAEYVLVPYADYMLLKFGDKEQAME
KIKDLTLISDILPTGFHGCVSAGVKPGSHVYIAGAGPVGR
CAAAGARLLGAACVIVGDQNPERLKLLSDAGFETIDLRNS
APLRDQIDQILGKPEVDCGVDAVGFEAHGLGDEANTETPN
GALNSLFDVVRAGGAIGIPGIYVGSDPDPVNKDAGSGRLH
LDFGKMWTKSIRIMTGMAPVINYNRHLTEAILWDQMPYLS
KVMNIEVITLDQAPDGYAKFDKGSPAKFVIDPHGMLKNKL
EHHHHHH

In some embodiments, the formaldehyde dismutase has an amino acid sequence having at least 70% amino acid residue identity with SEQ ID NO:3. In some embodiments, the formaldehyde dismutase comprises at last one or more, or all, of the following conserved domains:
(a) a NAD⁺-binding domain comprising one of the following conserved sequences:

(SEQ ID NO: 28)
GXGXXG,
(SEQ ID NO: 29)
GXGXXGX₁₈D,
(SEQ ID NO: 30)
GXGGXXGX₁₈D,
(SEQ ID NO: 31)
GXGGXXGX₁₉D,
(SEQ ID NO: 32)
GXGXVGX₅GX₄GAAXXIXXD;

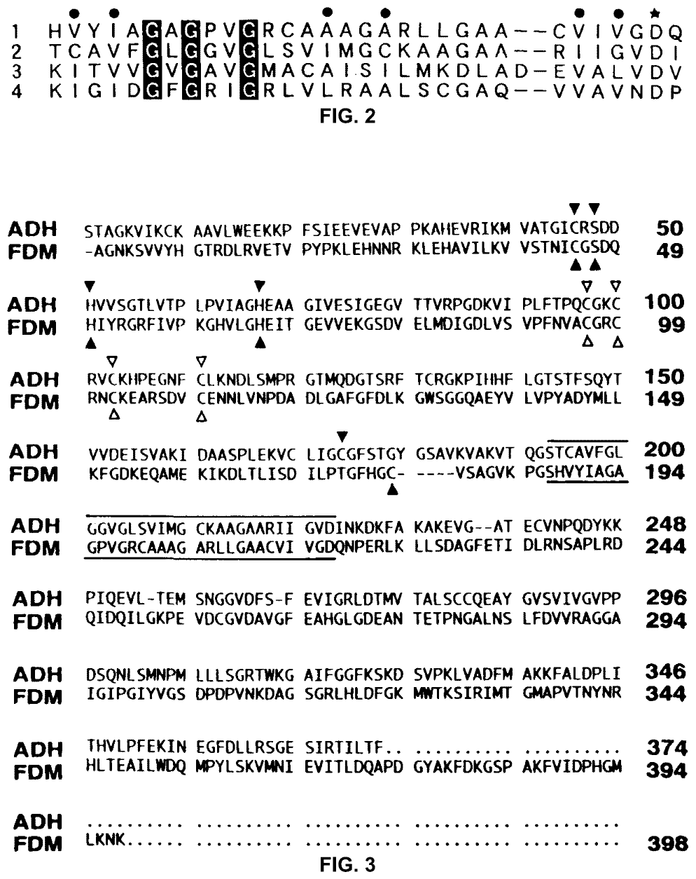

(b) ligands for binding a first catalytic zinc comprising one of the following conserved sequences: CXSXXHX₁₅H (SEQ ID NO:33) or CXSDXHX₃GX₄PX₅GH (SEQ ID NO:34); and,
(c) ligands for binding a second catalytic zinc comprising one of the following conserved sequences: CXXCXXCX₇C (SEQ ID NO:35) or CGXCRXCKX₆C (SEQ ID NO:36);
   wherein “X” is any amino acid residue. Certain of these conserved sequences are disclosed in FIGS. 2 and 3.

In some embodiments, the fusion protein comprises a methanol oxidase linked via a linker to a formate oxidase. In some embodiments, the fusion protein comprises a methanol oxidase linked via a linker to a formaldehyde dismutase. In some embodiments, the fusion protein comprises a formate oxidase linked via a linker to a formaldehyde dismutase. In some embodiments, the fusion protein comprises a methanol oxidase (i) linked via a first linker to a formate oxidase and (ii) linked via a second linker to a formaldehyde dismutase. In some embodiments, the fusion protein comprises a formate oxidase (i) linked via a first linker to a methanol oxidase and (ii) linked via a second linker to a formaldehyde dismutase. In some embodiments, the fusion protein comprises a formaldehyde dismutase (i) linked via a first linker to a methanol oxidase and (ii) linked via a second linker to a formate oxidase. In all embodiments, the fusion protein may comprise functional variants of the methanol oxidase, formate oxidase, and/or formaldehyde dismutase, wherein (as mentioned above) the functional variant comprises sequences that are at least 70% functionally equivalent.

In some embodiments, the fusion protein comprises the amino acid sequence of SEQ ID NO:41, 42, or 43.

A particular embodiment of a fusion protein comprises formate oxidase and methanol

(SEQ ID NO: 41)
MVQSHYDFVIVGGGTAGNTVAGRLAENPNVTVLVVEAGVA
NSADLPEITTPSNAMNLRGSKHDWAYKTTLVKRDDYERIE
KPNTRGKALGGSSSLNYFTWIPGCKPTFDRWAEYGGEEWT
WDPLVPYLRKSATYHDDTGLYNPELKKLGAGGPIPISHSE
LVEELEPFRENLIKAWKSTGKPFTENIYDGEMIGLNHCIS
TIYHGKRSGSFLFVKNRPNITIIPEVHSKNLIIDASNTAK
GVVVIDKEGNEHSFYATREVILSQGVFESPKLLMLSGVGP
RKELESNGIEVKVESRHVGQNLLDHPGVPFVLQVKDDICV
DDILMRQNEKNKAAHVQYQKDGSGPVGSGLLELVGFPRID
EYFEKDPLYRERKAANGGKDPFCPEGQPHFELDFVGMYGT
AFQWHFPTPKKGSHITIVVDLVRPVSEGGEVTLNSADPLE
QPKINLNFFADELDIVGMREGIRFTYDLLTKGDGFKDLVV
KEFPWEMPLDDDKEMRRAVLDRCQTAFHPCGTNRLSKNIE
QGVVDPALKVHGVKNLRVIDASIIPVIPDCRIQNSVYMIG
EKGADLIKAAHKDLYNGSGLVPRGSASMSDSEVNQEAKPE
VKPEVKPETHINLKVSDGSSEIFFKIKKTTPLRRLMEAFA
KRQGKEMDSLRFLYDGIRIQADQTPEDLDMEDNDIIEAHR
EQIGGHMGHPEEVDVIVCGGGPAGCVVAGRLAYADPTLKV
MLIEGGANNRDDPWVYRPGIYVRNMQRNGINDKATFYTDT
MASSYLRGRRSIVPCANILGGGSSINFQMYTRASASDWDD
FKTEGWTCKDLLPLMKRLENYQKPCNNDTHGYDGPIAISN
GGQIMPVAQDFLRAAHAIGVPYSDDIQDLTTAHGAEIWAK
YINRHTGRRSDAATAYVHSVMDVQDNLFLRCNARVSRVLF
DDNNKAVGVAYVPSRNRTHGGKLHETIVKARKMVVLSSGT
LGTPQILERSGVGNGELLRQLGIKIVSDLPGVGEQYQDHY
TTLSIYRVSNESITTDDFLRGVKDVQRELFTEWEVSPEKA
RLSSNAIDAGFKIRPTEEELKEMGPEFNELWNRYFKDKPD
KPVMFGSIVAGAYADHTLLPPGKYITMFQYLEYPASRGKI
HIKSQNPYVEPFFDSGEMNNKADFAPIRWSYKKTREVARR
MDAFRGELTSHHPRFHPASPAACKDIDIETAKQIYPDGLT
VGIHMGSWHQPSEPYKHDKVIEDIPYTEEDDKAIDDWVAD
HVETTWHSLGTCAMKPREQGGVVDKRLNVYGTQNLKCVDL
SICPDNLGTNTYSSALLVGEKGADLIAEELGLKIKTPHAP
VPHAPVPTGRPATQQVR.

A particular embodiment of a fusion protein comprises formate oxidase and methanol oxidase and having the following amino acid sequence:

(SEQ ID NO: 42)
MVQSHYDFVIVGGGTAGNTVAGRLAENPNVTVLVVEAGVA
NSADLPEITTPSNAMNLRGSKHDWAYKTTLVKRDDYERIE
KPNTRGKALGGSSSLNYFTWIPGCKPTFDRWAEYGGEEWT
WDPLVPYLRKSATYHDDTGLYNPELKKLGAGGPIPISHSE
LVEELEPFRENLIKAWKSTGKPFTENIYDGEMIGLNHCIS
TIYHGKRSGSFLFVKNRPNITIIPEVHSKNLIIDASNTAK
GVVVIDKEGNEHSFYATREVILSQGVFESPKLLMLSGVGP
RKELESNGIEVKVESRHVGQNLLDHPGVPFVLQVKDDICV
DDILMRQNEKNKAAHVQYQKDGSGPVGSGLLELVGFPRID
EYFEKDPLYRERKAANGGKDPFCPEGQPHFELDFVGMYGT
AFQWHFPTPKKGSHITIVVDLVRPVSEGGEVTLNSADPLE
QPKINLNFFADELDIVGMREGIRFTYDLLTKGDGFKDLVV
KEFPWEMPLDDDKEMRRAVLDRCQTAFHPCGTNRLSKNIE
QGVVDPALKVHGVKNLRVIDASIIPVIPDCRIQNSVYMIG
EKGADLIKAAHKDLYNGGHMGHPEEVDVIVCGGGPAGCVV
AGRLAYADPTLKVMLIEGGANNRDDPWVYRPGIYVRNMQR
NGINDKATFYTDTMASSYLRGRRSIVPCANILGGGSSINF
QMYTRASASDWDDFKTEGWTCKDLLPLMKRLENYQKPCNN
DTHGYDGPIAISNGGQIMPVAQDFLRAAHAIGVPYSDDIQ
DLTTAHGAEIWAKYINRHTGRRSDAATAYVHSVMDVQDNL
FLRCNARVSRVLFDDNNKAVGVAYVPSRNRTHGGKLHETI
VKARKMVVLSSGTLGTPQILERSGVGNGELLRQLGIKIVS
DLPGVGEQYQDHYTTLSIYRVSNESITTDDFLRGVKDVQR
ELFTEWEVSPEKARLSSNAIDAGFKIRPTEEELKEMGPEF
NELWNRYFKDKPDKPVMFGSIVAGAYADHILLPPGKYITM
FQYLEYPASRGKIHIKSQNPYVEPFFDSGFMNNKADFAPI
RWSYKKTREVARRMDAFRGELTSHHPRFHPASPAACKDID
IETAKQIYPDGLTVGIHMGSWHQPSEPYKHDKVIEDIPYT
EEDDKAIDDWVADHVETTWHSLGTCAMKPREQGGVVDKRL
NVYGTQNLKCVDLSICPDNLGINTYSSALLVGEKGADLIA
EELGLKIKTPHAPVPHAPVPTGRPATQQVR.

A particular embodiment of a fusion protein comprises formate oxidase and methanol oxidase with a SUMO tag and having the following amino acid sequence:

(SEQ ID NO: 43)
MVQSHYDFVIVGGGTAGNTVAGRLAENPNVTVLVVEAGVA
NSADLPEITTPSNAMNLRGSKHDWAYKTTLVKRDDYERIE
KPNTRGKALGGSSSLNYFTWIPGCKPTFDRWAEYGGEEWT
WDPLVPYLRKSATYHDDTGLYNPELKKLGAGGPIPISHSE
LVEELEPFRENLIKAWKSTGKPFTENIYDGEMIGLNHCIS
TIYHGKRSGSFLFVKNRPNITIIPEVHSKNLIIDASNTAK
GVVVIDKEGNEHSFYATREVILSQGVFESPKLLMLSGVGP
RKELESNGIEVKVESRHVGQNLLDHPGVPFVLQVKDDICV
DDILMRQNEKNKAAHVQYQKDGSGPVGSGLLELVGFPRID
EYFEKDPLYRERKAANGGKDPFCPEGQPHFELDFVGMYGT
AFQWHFPTPKKGSHITIVVDLVRPVSEGGEVTLNSADPLE
QPKINLNFFADELDIVGMREGIRFTYDLLTKGDGFKDLVV
KEFPWEMPLDDDKEMRRAVLDRCQTAFHPCGTNRLSKNIE
QGVVDPALKVHGVKNLRVIDASIIPVIPDCRIQNSVYMIG
EKGADLIKAAHKDLYNGGSAGNKSVVYHGTRDLRVETVPY
PKLEHNNRKLEHAVILKVVSTNICGSDQHIYRGRFIVPKG
HVLGHEITGEVVEKGSDVELMDIGDLVSVPFNVACGRCRN
CKEARSDVCENNLVNPDADLGAFGFDLKGWSGGQAEYVLV
PYADYMLLKFGDKEQAMEKIKDLTLISDILPTGFHGCVSA
GVKPGSHVYIAGAGPVGRCAAAGARLLGAACVIVGDQNPE
RLKLLSDAGFETIDLRNSAPLRDQIDQILGKPEVDCGVDA
VGFEAHGLGDEANTETPNGALNSLFDVVRAGGAIGIPGIY
VGSDPDPVNKDAGSGRLHLDFGKMWTKSIRIMTGMAPVTN
YNRHLTEAILWDQMPYLSKVMNIEVITLDQAPDGYAKFDK
GSPAKFVIDPHGMLKNKGSGLVPRGSASMSDSEVNQEAKP
EVKPEVKPETHINLKVSDGSSEIFFKIKKTTPLRRLMEAF
AKRQGKEMDSLRFLYDGIRIQADQTPEDLDMEDNDIIEAH
REQIGGHMGHPEEVDVIVCGGGPAGCVVAGRLAYADPTLK
VMLIEGGANNRDDPWVYRPGIYVRNMQRNGINDKATFYTD
TMASSYLRGRRSIVPCANILGGGSSINFQMYTRASASDWD
DFKTEGWTCKDLLPLMKRLENYQKPCNNDTHGYDGPIAIS
NGGQIMPVAQDFLRAAHAIGVPYSDDIQDLTTAHGAEIWA
KYINRHTGRRSDAATAYVHSVMDVQDNLFLRCNARVSRVL
EDDNNKAVGVAYVPSRNRTHGGKLHETIVKARKMVVLSSG
TLGTPQILERSGVGNGELLRQLGIKIVSDLPGVGEQYQDH
YTTLSIYRVSNESITTDDFLRGVKDVQRELFTEWEVSPEK
ARLSSNAIDAGFKIRPTEEELKEMGPEFNELWNRYFKDKP
DKPVMFGSIVAGAYADHILLPPGKYITMFQYLEYPASRGK
IHIKSQNPYVEPFFDSGFMNNKADFAPIRWSYKKTREVAR
RMDAFRGELTSHHPRFHPASPAACKDIDIETAKQIYPDGL
TVGIHMGSWHQPSEPYKHDKVIEDIPYTEEDDKAIDDWVA
DHVETTWHSLGTCAMKPREQGGVVDKRLNVYGTQNLKCVD
LSICPDNLGINTYSSALLVGEKGADLIAEELGLKIKTPHA
PVPHAPVPTGRPATQQVR.

In some embodiments, the linker, or first linker and/or second linker, is a covalent bond, or one or more amino acid residues. In some embodiments, the linker is at least about 1, 2, 3, 4, 5, 6, 7. 8, 9, or 10 amino acid residues in length. In some embodiments, the linker is up to about 100, 200, 300, 400, 500, 600, 700, 800, 900, or 1,000 amino acid residues in length. In some embodiments, the linker is from about 1, 2, 3, 4, 5, 6, 7. 8, 9, or 10, to about 100, 200, 300, 400, 500, 600, 700, 800, 900, or 1,000 amino acid residues in length.

In some embodiments, the linker comprises a tag, such as a Small Ubiquitin-like Modifier (SUMO) tag. In some embodiments, the linker comprises an amino acid sequence as follows: GGH,

(SEQ ID NO: 44)
GSGLVPRGSASMSDSEVNQEAKPEVKPEVKPETHINLKVS
DGSSEIFFKIKKTTPLRRLMEAFAKRQGKEMDSLRFLYDG
IRIQADQTPEDLDMEDNDIIEAHREQIGGH,
or
(SEQ ID NO: 45)
GGSAGNKSVVYHGTRDLRVETVPYPKLEHNNRKLEHAVIL
KVVSTNICGSDQHIYRGRFIVPKGHVLGHEITGEVVEKGS
DVELMDIGDLVSVPENVACGRCRNCKEARSDVCENNLVNP
DADLGAFGFDLKGWSGGQAEYVLVPYADYMLLKFGDKEQA
MEKIKDLTLISDILPTGFHGCVSAGVKPGSHVYIAGAGPV
GRCAAAGARLLGAACVIVGDQNPERLKLLSDAGFETIDLR
NSAPLRDQIDQILGKPEVDCGVDAVGFEAHGLGDEANTET
PNGALNSLFDVVRAGGAIGIPGIYVGSDPDPVNKDAGSGR
LHLDFGKMWTKSIRIMTGMAPVINYNRHLTEAILWDQMPY
LSKVMNIEVITLDQAPDGYAKFDKGSPAKFVIDPHGMLKN
KGSGLVPRGSASMSDSEVNQEAKPEVKPEVKPETHINLKV
SDGSSEIFFKIKKTTPLRRLMEAFAKRQGKEMDSLRFLYD
GIRIQADQTPEDLDMEDNDIIEAHREQIGGH.

The present invention provides for a host cell comprising a polynucleotide encoding a methanol oxidase, formate oxidase, and/or formaldehyde dismutase, or a fusion protein of the present invention, each operatively linked to separate promoters or one promoter.

In some embodiments, the polynucleotide is a vector capable of stably residing in the host cell. In some embodiments, the vector is a plasmid. In some embodiments, the vector is an expression vector. In some embodiments, the promoter is an inducible promoter or constitutive promoter. In some embodiments, the promoter is heterologous to the enzyme to which it is operably linked to.

The present invention provides for a method of producing a methanol oxidase, formate oxidase, and/or formaldehyde dismutase comprising: (a) providing a host cell of the present invention in a nutrient medium, and (b) culturing or growing the host cell in the nutrient medium such that the methanol oxidase, formate oxidase, and/or formaldehyde dismutase is expressed or produced. In some embodiments, the method further comprises: (c) separating the methanol oxidase, formate oxidase, and/or formaldehyde dismutase from the rest of the host cell and/or nutrient medium. In some embodiments, the (c) separating step comprises isolating or purifying the methanol oxidase, formate oxidase, and/or formaldehyde dismutase. In some embodiments, the methanol oxidase, formate oxidase, and/or formaldehyde dismutase comprise a tag, such a histidine tag (such as a C-terminal six histidine tag), and the isolating or purifying comprises using an affinity column based on the affinity of the tag to the affinity column. In some embodiments, the tag comprises the amino acid sequence MGSSHHHHHHGGS (SEQ ID NO:46). The tag can be located at the N-terminal, C-terminal, within the enzyme or fusion protein, or within a linker of the fusion protein. In some embodiments, the tag comprises the amino acid sequence of a SUMO tag or a tag that aids solubility. The SUMO tag aids in either solubility or protein folding (or both) of the methanol oxidase protein. In some embodiments, the tag that aids solubility is amino acid sequence that aids in the increasing the solubility of the enzyme or fusion protein, including, but not limited to, maltose binding protein (MBP), glutathione-S-transferase (GST), and thioredoxin (TRX).

Any prokaryotic or eukaryotic host cell may be used in the present method so long as it remains viable after being transformed with the polynucleotide. Generally, although not necessarily, the host microorganism is bacterial. In some embodiments, the host cell is a Gram negative bacterium. In some embodiments, the host cell is of the phylum Proteobactera. In some embodiments, the host cell is of the class Gammaproteobacteria. In some embodiments, the host cell is of the order Enterobacteriales. In some embodiments, the host cell is of the family Enterobacteriaceae. Examples of bacterial host cells include, without limitation, those species assigned to the Escherichia (such as E. coli), Enterobacter, Azotobacter, Erwinia, Bacillus, Pseudomonas (such as P. putida), Klebsiella, Proteus, Salmonella, Serratia, Shigella, Rhizobia, Vitreoscilla, and Paracoccus genera. The host cell is not adversely affected by the transduction of the necessary nucleic acid sequences, and/or the subsequent expression of the proteins (i.e., enzymes). Suitable eukaryotic cells include, but are not limited to, fungal, insect or mammalian cells. Suitable fungal cells are yeast cells, such as yeast cells of the Saccharomyces (such as S. cerevisae) or Rhodosporidiur (such as R. toruloides) genera.

The hydrogen peroxide can be used in the paper and pulp industry, in the production of sodium percarbonate and sodium perborate for laundry detergent, in the production of propylene oxide, in the waste water treatment industry, and for mining. Hydrogen peroxide is also potentially useful for ethylene oxide production. The process should enable economic production of hydrogen peroxide in smaller batches on site for hydrogen peroxide consumers. This reduces the need to ship hydrogen peroxide long distance and can be stored on site. In the long run the process might produce hydrogen peroxide cheaper than the current anthraquinone process.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Materials and Methods:

### Plasmids:

Each plasmid has a constitutively expressed kanamycin resistance gene for selection, a pBR322 origin of replication for plasmid maintenance, a lacI gene that expresses constitutively for T7 promoter repression, a F1 origin for potential single stranded DNA generation, a T7 promoter for overexpression of the gene of interest, and a T7 terminator to terminate mRNA production. Genes for the hydrogen peroxide system were cloned between the T7 promoter and T7 terminator as appropriate for expression by Isopropyl β-D-1-thiogalactopyranoside (IPTG) induction. This resulted in a total of 3 plasmids named pHP2, pHP23, pHP30, each with one gene from the hydrogen peroxide generating system in it.

Plasmid pHP2 expresses a formate oxidase from Schwanniomyces vanrijiae with a C-terminal six histidine (6×His) tag for purification. Plasmid pHP23 uses a T7 promoter to express formaldehyde dismutase from Pseudomonas putida with a C-terminal 6×His tag for purification. Plasmid pHP30 uses a T7 promoter to express methanol oxidase from Phanerochaete chrysosporium with a N-terminal 6×His followed by a small ubiquitin-like modifier (SUMO) tag followed by the coding sequence of methanol oxidase.

### Plasmid Expressions:

All glassware, pipet tips, media, etc. used in cell culture are either autoclaved or sterile filtered prior to use or purchased pre-sterilized. Unless otherwise noted, kanamycin supplementation is used at a concentration of 50 μg/mL. Plasmids pHP2, pHP23, and pHP30 are transformed into E. coli BL21DE3* cells using the KCM method 1. Briefly, on day one BL21DE3* cells are streaked from a glycerol stock onto lysogeny broth (LB) agar plates (1.0% weight to volume (w/v) tryptone, 0.5% w/v yeast extract, 1.0% w/v sodium chloride, 1.5% w/v agar), and placed in a 37° C. incubator overnight.

On day two a single colony was picked into 10 mL of LB liquid media (as LB agar, but without the 1.5% w/v agar) in a glass test tubes and incubated overnight (approximately sixteen hours) at 37° C., shaking at 250 rotation per minute (RPM).

On day three, 0.5 mL of the day two overnight is used to inoculate 50 mL of fresh LB liquid media in a 250 mL baffled flask and allowed to grow at 37° C. shaking at 250 RPM until the cells reach an optical density at 600 nm of approximately 0.35. The culture is then chilled on ice for 20 minutes prior to centrifugation at 8,000 relative centrifugal force (RCF) for 8 minutes in conical plastic tubes at 4° C. Supernatant is then decanted, and the cells are resuspended in 5 mL TSS media (1.0% w/v tryptone, 0.5% w/v yeast extract, 1.0% w/v sodium chloride, 10% w/v polyethylene glycol with average mol weight 3,350, 5% dimethyl sulfoxide v/v, 20 mM MgCl₂). 100 μL aliquots of the TSS media cell mixture are then pipetted into 0.6 mL plastic tubes with caps. 10 ng of appropriate plasmid is then added to the TSS media cell mixture containing tubes, resulting in 3 tubes, each with one plasmid. 2×KCM (0.06 M KCl, 0.2 M CaCl₂, 0.1 M MgCl₂) is added and mixed into the 0.6 mL tubes. Tubes are incubated on ice for 20 minutes before heat shocking at 42° C. for 90 seconds. The tubes are returned to ice for 2 minutes before adding 200 μL of terrific broth (TB) and are incubated at 37° C. for 1 hour. TB is composed of 1.2% w/v tryptone, 2.4% w/v yeast extract, 0.4% v/v glycerol, 72 mM K₂HPO₄, and 17 mM KH₂PO₄. The full tube liquid volume, approximately 400 μL, is then spread onto LB agar plates supplemented with kanamycin using glass beads. Plates are then incubated at 37° C. overnight, approximately sixteen hours. At this point there are 3 plates, each with cells only containing either pHP2, pHP23, or pHP30.

On day four, single colonies are picked into 10 mL LB media supplemented with kanamycin. Cultures are allowed to grow overnight, approximately sixteen hours, at 37° C. shaking at 250 RPM.

On day five, 0.5 mL of saturated cultures from day four are used to inoculate 50 mL of fresh TB media with kanamycin, in a 250 mL baffled flasks. At this point there are 3 baffled flasks, each with cells only containing either pHP2, pHP23, or pHP30. Cells are then grown shaking at 250 RPM at 37° C. until they reach an optical density at 600 nm of 0.8. Isopropyl-β-D-thiogalactoside (IPTG) is added at a final concentration of 1 mM to each tube and the temperature is reduced to 18° C. Shaking otherwise remained the same. Cells are allowed to grow for 24 hours at 18° C. before harvesting in a centrifuge at 8,000 RCF for 8 minutes in conical plastic tubes at 4° C. The supernatant is decanted, and the cell pellets ire frozen at −20° C. until purification.

### Enzyme Purification:

All column steps are carried out in a 4° C. room. Purification buffers (lysis buffer, wash buffer, elution buffer, dialysis buffer 1, dialysis buffer 2) are prepared using distilled and deionized water and are chilled to 4° C. before use. Lysis buffer consists of 50 mM sodium phosphate buffer pH 7.2, 25 mM imidazole, 1 mM β-mercaptoethanol (BME), 1 mg/mL lysozyme and 0.1 mg/mL of DNAse. Wash buffer consists of 50 mM sodium phosphate buffer pH 7.2, 25 mM imidazole, and 1 mM BME. Elution buffer consists of 50 mM sodium phosphate buffer pH 7.2, 200 mM imidazole and 1 mM BME. Dialysis buffer 1 consists of 50 mM sodium phosphate buffer pH 7.2 with 1 mM tris(2-carboxyethyl)phosphine hydrochloride (TCEP). Dialysis buffer 2 consists of 50 mM sodium phosphate buffer pH 7.2. Cell pellets are resuspended in 5 mL of lysis buffer and sonicated on ice to disrupt the cell membrane. The sonication protocol uses a cycle of 5 seconds sonicating, 10 seconds without sonicating, and repeated 12 times per cell pellet with enough amplitude to disrupt the cells, in this cast 30% power. The disrupted cells are then clarified by centrifugation at 15,000 RCF for forty five minutes at 4° C. Concurrently columns containing approximately 1 mL of Ni-NTA agarose beads are washed with 10 mL wash buffer. Flow through is discarded. Upon completion of centrifugation, the clarified supernatant from the lysed cells is applied to the column and allowed to flow through. The flow through is discarded. The columns are then washed with 40 mL of wash buffer. Flow through is discarded. 15 mL of elution buffer is then added to the columns, and flow through is collected in 10,000 molecular weight cut off (MWCO) conical spin filters. Samples are centrifuged at 6,000 RCF at 4° C. until concentrated to approximately 700 μL. The concentrated protein is transferred to a dialysis cassette with a 3,500 MWCO membrane. Flow through from the concentration step is discarded. Concentrated proteins are dialyzed in dialysis buffer 1 overnight, approximately sixteen hours. Concentrated proteins are then dialyzed in dialysis buffer 2 for four hours. Cells are then used immediately for assays. At this stage there are three individual concentrated and purified proteins.

### Protein Assays:

Protein assays are conducted at room temperature. Hydrogen peroxide concentrations are tested with a commercially available coulometric kit that could detect between 0 and 100 mg/L hydrogen peroxide.

Purified methanol oxidase, still with 6×His tag and SUMO tag, is added to 50 mM sodium phosphate buffer, pH 7.2 with and without 270 mM methanol with a final volume of 500 μL in a 2 mL plastic tube. The tube is vortexed and allowed to sit for one hour before testing for hydrogen peroxide. In the condition with methanol, the commercial kit detects between 1 and 100 mg/L hydrogen peroxide. In the no methanol condition, the kit detects 0 mg/L hydrogen peroxide.

Purified formate oxidase, still with 6×His tag, is added to 50 mM sodium phosphate buffer, pH 7.2 with and without 270 mM sodium formate with a final volume of 500 μL in a 2 mL plastic tube. The tube is vortexed and allowed to sit for one hour before testing for hydrogen peroxide. In the condition with sodium formate, the commercial kit detects between 1 and 100 mg/L hydrogen peroxide. In the no sodium formate condition, the kit detects 0 mg/L hydrogen peroxide.

Purified formaldehyde dismutase, still with 6×His tag, is added to 50 mM sodium phosphate buffer, pH 7.2 with and without 30 mM formaldehyde with a final volume of 500 μL in a 2 mL plastic tube. The tubes are vortexed and allowed to sit for one hour before testing for hydrogen peroxide. In both conditions 0 mg/L hydrogen peroxide is detected.

Purified formaldehyde dismutase, still with 6×His tag, and methanol oxidase, still with 6×His tag and SUMO tag, are added to 50 mM sodium phosphate buffer, pH 7.2 with and without 30 mM formaldehyde with a final volume of 500 μL in a 2 mL plastic tube. The tubes are vortexed and allowed to sit for one hour before testing for hydrogen peroxide. In the condition with formaldehyde, the commercial kit detects between 1 and 100 mg/L hydrogen peroxide. In the condition without the formaldehyde, 0 mg/L hydrogen peroxide is detected.

Purified formaldehyde dismutase, still with 6×His tag, and formate oxidase, still with 6×His tag, are added to 50 mM sodium phosphate buffer, pH 7.2 with and without 30 mM formaldehyde with a final volume of 500 μL in a 2 mL plastic tube. The tubes are vortexed and allowed to sit for one hour before testing for hydrogen peroxide. In the condition with formaldehyde, the commercial kit detects between 1 and 100 mg/L hydrogen peroxide. In the condition without the formaldehyde, 0 mg/L hydrogen peroxide is detected.

Purified formaldehyde dismutase, still with 6×His tag, and formate oxidase, still with 6×His tag, and methanol oxidase, still with 6×His tag and SUMO tag, are added to 50 mM sodium phosphate buffer, pH 7.2 with and without 270 mM methanol with a final volume of 500 μL in a 2 mL plastic tube. The tubes are vortexed and allowed to sit for one hour before testing for hydrogen peroxide. In the condition with methanol, the commercial kit detects between 1 and 100 mg/L hydrogen peroxide. In the condition without the methanol, 0 mg/L hydrogen peroxide is detected.

### REFERENCES CITED

1. Eiben, C. B. et al. Mevalonate pathway promiscuity enables noncanonical terpene production. ACS Synth. Biol. (2019).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for producing hydrogen peroxide comprising:
(a) contacting formaldehyde with a methanol oxidase bound with a flavin adenine dinucleotide (FAD) cofactor or a formate oxidase bound with a FAD cofactor, wherein the formaldehyde is oxidized to a formate and the FAD is reduced to FADH₂; and
(b) contacting oxygen with the FADH₂, wherein the oxygen is reduced to hydrogen peroxide and the FADH₂ is oxidized to FAD,
wherein:
the methanol oxidase comprises a Phanerochaete chrysosporium methanol oxidase, and
the formate oxidase comprises a Schwanniomyces vanrijiae formate oxidase.

2. The method of claim 1, further comprising an antecedent step of:
(c) contacting methanol with the methanol oxidase bound with the FAD cofactor, or with the formate oxidase bound with the FAD cofactor, wherein the methanol is oxidized to the formaldehyde and the FAD cofactor is reduced to FADH₂.

3. The method of claim 1 wherein:
step (a) comprises contacting the formaldehyde with the methanol oxidase bound with the FAD cofactor, wherein the formaldehyde is oxidized to formate and the FAD is reduced to FADH₂.

4. The method of claim 1 wherein:
step (a) comprises contacting the formaldehyde with the formate oxidase bound with the FAD cofactor, wherein the formaldehyde is oxidized to formate and the FAD is reduced to FADH₂.

5. The method of claim 2 wherein:
step (a) comprises contacting the formaldehyde with the methanol oxidase bound with the FAD cofactor, wherein the formaldehyde is oxidized to formate and the FAD is reduced to FADH₂, and
step (c) comprises contacting the methanol with the methanol oxidase bound with the FAD cofactor, wherein the methanol is oxidized to formaldehyde and the FAD cofactor is reduced to FADH₂.

6. The method of claim 2 wherein:
step (a) comprises contacting the formaldehyde with the methanol oxidase bound with the FAD cofactor, wherein the formaldehyde is oxidized to formate and the FAD is reduced to FADH₂, and
step (c) comprises contacting the methanol with the formate oxidase bound with a FAD cofactor, wherein the methanol is oxidized to formaldehyde and the FAD cofactor is reduced to FADH₂.

7. The method of claim 2 wherein:
step (a) comprises contacting the formaldehyde with the formate oxidase bound with the FAD cofactor, wherein the formaldehyde is oxidized to formate and the FAD is reduced to FADH₂, and
step (c) comprises contacting the methanol with the methanol oxidase bound with the FAD cofactor, wherein the methanol is oxidized to formaldehyde and the FAD cofactor is reduced to FADH₂.

8. The method of claim 2 wherein:
step (a) comprises contacting the formaldehyde with the formate oxidase bound with the FAD cofactor, wherein the formaldehyde is oxidized to formate and the FAD is reduced to FADH₂, and
step (c) comprises contacting the methanol with the formate oxidase bound with a FAD cofactor, wherein the methanol is oxidized to formaldehyde and the FAD cofactor is reduced to FADH₂.

9. The method of claim 1 wherein the methanol oxidase and the formate oxidase are combined in a fusion protein.

10. The method of claim 2 wherein the methanol oxidase and the formate oxidase are combined in a fusion protein.

11. The method of claim 1, further comprising the subsequent step of:
(i) contacting the formaldehyde with a formaldehyde dismutase, wherein the formaldehyde is converted to methanol and formate,
wherein the formaldehyde dismutase comprises a Pseudomonas putida formaldehyde dismutase.

12. The method of claim 11, further comprising:
(ii) contacting the hydrogen peroxide with a catalase, wherein the hydrogen peroxide is reduced to water.

13. The method of claim 12, wherein the catalase comprises KatE, KatG or a hydroperoxidase.

14. The method of claim 11, further comprising:
(i) contacting the hydrogen peroxide with a peroxidase, wherein the hydrogen peroxide is reduced to water and the peroxidase is activated with an oxidized active site, and
(ii) contacting an organic molecule with the activated peroxidase, wherein the organic molecule is oxidized.

15. The method of claim 14, wherein the organic molecule is a compound selected from methanol, formaldehyde, and formate.

16. The method of claim 11 wherein the methanol oxidase, the formate oxidase and the formaldehyde dismutase are combined in a fusion protein.

17. The method of claim 11 wherein:
step (a) comprises contacting the formaldehyde with the methanol oxidase bound with the FAD cofactor, wherein the formaldehyde is oxidized to formate and the FAD is reduced to FADH₂, and
further comprising the antecedent step of:
(c) contacting the methanol with the methanol oxidase bound with the FAD cofactor, wherein the methanol is oxidized to formaldehyde and the FAD cofactor is reduced to FADH₂.

18. The method of claim 11 wherein:
step (a) comprises contacting the formaldehyde with the methanol oxidase bound with the FAD cofactor, wherein the formaldehyde is oxidized to formate and the FAD is reduced to FADH₂, and
further comprising the antecedent step of:
step (c) contacting the methanol with the formate oxidase bound with a FAD cofactor, wherein the methanol is oxidized to formaldehyde and the FAD cofactor is reduced to FADH₂.

19. The method of claim 11 wherein:
step (a) comprises contacting the formaldehyde with the formate oxidase bound with the FAD cofactor, wherein the formaldehyde is oxidized to formate and the FAD is reduced to FADH₂, and
step (c) comprises contacting the methanol with the methanol oxidase bound with the FAD cofactor, wherein the methanol is oxidized to formaldehyde and the FAD cofactor is reduced to FADH₂.

20. The method of claim 11 wherein:
step (a) comprises contacting the formaldehyde with the formate oxidase bound with the FAD cofactor, wherein the formaldehyde is oxidized to formate and the FAD is reduced to FADH₂, and
step (c) comprises contacting the methanol with the formate oxidase bound with a FAD cofactor, wherein the methanol is oxidized to formaldehyde and the FAD cofactor is reduced to FADH₂.
